(19) United States
(12) Patent Application Publication
(10) Pub. No.: US 2026/0240443 A1
(43) Pub. Date: Aug. 20, 2026

(54) HEART RATE SENSOR AND METHOD FOR MEASURING A HEART RATE

(71) Applicant: Jawsaver B.V., Utrecht (NL)

(72) Inventors: Selina Tirtajana, Utrecht (NL); Gabriel Enrique Saenz, Utrecht (NL)

(73) Assignee: Jawsaver B.V., Utrecht (NL)

(21) Appl. No.: 19/055,302

(22) Filed: Feb. 17, 2025

(51) Int. Cl. A61B 5/024; A61B 5/00; A61B 5/11

(52) U.S. Cl. CPC A61B 5/02444; A61B 5/02405; A61B 5/1126; A61B 5/6817; A61B 5/721; A61B 5/7239; A61B 2560/0462; A61B 2562/0223; A61B 2562/164

(57) ABSTRACT

The current invention relates to a heart rate sensor for determining a heart rate. The current invention further relates to a method for determining a heart rate wherein use is made of a heart rate sensor according to the invention. The heart rate sensor comprises an in-ear device having an in-ear portion with a distal portion, a magnet, and a magnetic sensor. The heart rate sensor further comprises a processor configured to determine the heart rate based on a measured change in magnetic flux density caused by movement of the magnet relative to the magnetic sensor in the ear canal.

### FIELD OF THE INVENTION

The current invention relates to a heart rate sensor for determining a heart rate. The current invention further relates to a method for determining a heart rate wherein use is made of a heart rate sensor according to the invention.

### BACKGROUND OF THE INVENTION

Wearable devices for monitoring physiological parameters, such as a heart rate, are known and offer benefits for the user due to their portability and user friendly design. These devices allow for real time monitoring of the physiological parameters which allow these devices to be used as assessments aids in detecting events, tracking clinical conditions, and enabling targeted healthcare interventions, especially for chronic conditions like cardiovascular diseases, diabetes and stress.

In-ear wearables allow for a more stable placement, reduced motion artifacts and less susceptibility to peripheral blood flow issues. The unobstrusive nature of in-ear wearables, which may be integrated into earplugs or hearing aids, allow for improved user comfort and acceptance.

Additionally, in-ear wearables allow, given their closer proximity to the brain, for the integration of a sensing and monitoring system with a stimulation modality to create a closed-loop biofeedback or neuromodulation system.

Currently, photoplethysmography (PPG) is the most commonly used technology in wearables to estimate heart rate and other physiological parameters. PPG devices optically measure volumetric changes in blood vessels by analyzing light absorption using a light source, such as an LED, to emit light into the skin and a photodetector to measure the light that is either reflected or transmitted through the tissue. Changes in light absorption may correspond to pulsatile blood flow, allowing determining of heart rate and other physiological parameters. A downside of these devices is a need for a larger power source due to the presence of the LED and photodetector. In addition, PPG based devices are less effective for individuals with darker skin tones.

WO 2023/099429 A1 discloses a jaw movement tracking system comprising an in ear device having a magnet and a magnetic sensor. Jaw movement is tracked by determining the relative movement of the magnet to the magnetic sensor which may be used to determine the occurrence of an event such as bruxism.

### OBJECT OF THE INVENTION

The invention aims to provide an improved heart rate sensor. It is a further aim of the invention to provide a heart rate sensor that allows to overcome one or more of the downsides mentioned above.

### SUMMARY OF THE INVENTION

Embodiments of the present disclose provide a heart rate sensor for determining a heart rate, comprising:
an in-ear device comprising:
   an in-ear portion configured to contact an ear canal of an user wherein the in-ear portion comprises a distal portion made from a flexible material such that the distal portion moves, e.g. changes shape, in response to a deformation or vibration of the ear canal when the in-ear portion is placed in the ear canal;
   a magnet;
   a magnetic sensor for measuring a change in magnetic flux density caused by movement of the magnet relative to the magnetic sensor; and
a processor connected to the magnetic sensor for receiving a signal representing the measured change in magnetic flux density;
   wherein either the magnet or the magnetic sensor is provided in the distal portion such that a movement of the distal portion resulting from a movement or vibration of the ear canal causes a change in the magnetic flux density measured by the magnetic sensor,
   wherein the processor is configured to:
determine a movement of the magnet relative to the magnetic sensor based on the measured change in magnetic flux density;
determine a heart rate of the user, e.g. by first determining a heart beat waveform of the user, based on the determined movement of the magnet relative to the magnetic sensor; and
output a signal representing the determined heart rate of the user.

In embodiments of the disclosure, the processor is configured to determine the heart rate or the heart beat waveform of the user based on the determined movement of the magnet relative to the magnetic sensor by obtaining a waveform signal from the determined movement of the magnet relative to the magnetic sensor and comparing the waveform signal to a cardiac cycle.

In further embodiments of the disclosure, the processor is further configured to:
determine a movement of the magnet relative to the magnetic sensor based on the measured change in magnetic flux density in an x-direction, y-direction, and/or z-direction of the ear canal;
determine the waveform signal in the x-direction, y-direction, and/or z-direction;
determine a cardinal direction of the waveform signal based on the determined waveform signal in the x-direction, y-direction, and/or z-direction; and
compare the waveform signal in the cardinal direction to the cardiac cycle.

In further embodiments of the disclosure, the processor is configured to determine the cardinal direction of the waveform signal by determining a direction of the waveform signal where an amplitude of the waveform signal is above a threshold value.

In embodiments of the disclosure, the processor is configured to determine the heart rate or the heart beat waveform of the user based on the determined movement of the magnet relative to the magnetic sensor by using an empirical model or a regression model modeling the relation between the heart rate and the determined movement.

In embodiments of the disclosure, the processor is configured to extract the waveform signal by filtering the waveform signal from the determined movement.

In further embodiments of the disclosure, the sensor further comprises a movement sensor for measuring motion artifacts caused by movement of the in-ear device, wherein the movement sensor is operatively connected to the processor for providing a signal to the processor representative of the measured motion artifacts, wherein the processor is configured to:
remove the motion artifacts from the waveform signal.

In embodiments of the disclosure, the in-ear device comprises a middle portion provided between the distal portion and a proximal portion, wherein the other one of the magnet or the magnetic sensor is provided in the proximal portion, and wherein the middle portion is configured to allow independent movement of the distal portion relative to the proximal portion.

In embodiments of the disclosure, the processor is further configured to determine one or more-but not limited to-of an inter-beat interval, a heart rate variability, a derivative of the heart rate variability, and spectral signals of the heart rate variability based on the determined movement of the magnet relative to the magnetic sensor, and to output a signal representing the determined one or more of-but not limited to-the inter-beat interval, the heart rate variability, the derivative of the heart rate variability, and spectral signals of the heart rate variability.

In embodiments of the disclosure, the magnetic sensor is a hall effect sensor.

In embodiments of the disclosure, the shape conforming material is a suitable material that is configured to support the magnets or the magnetic sensors in the ear canals and to conform the shape thereof to the shape of the ear canal, while still allowing movement of the magnets or the magnetic sensors in response to the movement or vibration of the ear canal.

In embodiments of the disclosure, the in-ear device further comprises a support portion which, when the in-ear device is placed in the ear canal, extends out of the ear canal to support the in-ear device on the ear.

Embodiments of the present disclosure further relate to a method for determining a heart rate wherein use is made of the heart rate sensor according to the present invention.

In embodiments of the disclosure, the method comprises:
determining a movement of the magnet relative to the magnetic sensor based on the measured change in magnetic flux density;
determining a heart rate of the user, e.g. by first determining a heart beat waveform of the user, based on the determined movement of the magnet relative to the magnetic sensor; and
outputting a signal representing the determined heart rate of the user.

In embodiments of the disclosure, the method further comprises:
determining the heart rate of the user based on the determined movement of the magnet relative to the magnetic sensor by obtaining a waveform signal from the determined movement of the magnet relative to the magnetic sensor; and
comparing the waveform signal to a cardiac cycle.

In embodiments of the disclosure, the method further comprises:
determining a movement of the magnet relative to the magnetic sensor based on the measured change in magnetic flux density in an x-direction, y-direction, and/or z-direction of the ear canal;
determining the waveform signal in the x-direction, y-direction, and/or z-direction;
determining a cardinal direction of the waveform signal based on the determined waveform signal in the x-direction, y-direction, and/or z-direction; and
comparing the waveform signal in the cardinal direction to the cardiac cycle.

In further embodiments of the disclosure, the method further comprises determining the cardinal direction of the waveform signal by determining a direction of the waveform signal where an amplitude of the waveform signal is above a threshold value.

### BRIEF DESCRIPTION OF THE DRAWINGS

The invention will be explained below with reference to the drawing in which:

FIG. 1 shows a schematic view of an embodiment of a heart rate sensor in an ear canal of the user;

FIG. 2 shows a schematic view of another embodiment of a heart rate sensor;

FIG. 3 shows an in-ear portion from various sides;

FIG. 4 shows a close-up of a distal portion of the in-ear device; and

FIG. 5 shows a cross-section of an embodiment of the distal portion.

### DESCRIPTION OF THE EMBODIMENTS

The auditory canal of a person, e.g. a user of the heart rate sensor, is richly perfused via various subdivisions of internal and external carotid. Blood flow travels as a pulse wave through the blood vessels in the ear canal following each heartbeat. When the heart pumps, blood pressure rises, and the diameter of the arteries expands as a result of this pressure wave on the vessel walls. This motion of the vessel walls may propagate through the tissue, causing vibrations and/or movements of the skin in the ear canal. For example, the pulse of one of the larger branches, the superficial temporal artery, may be felt around the tragus. For example, the in-ear portion 3 of the heart rate sensor 1 may allow measurement of the heart rate via the superficial temporal artery.

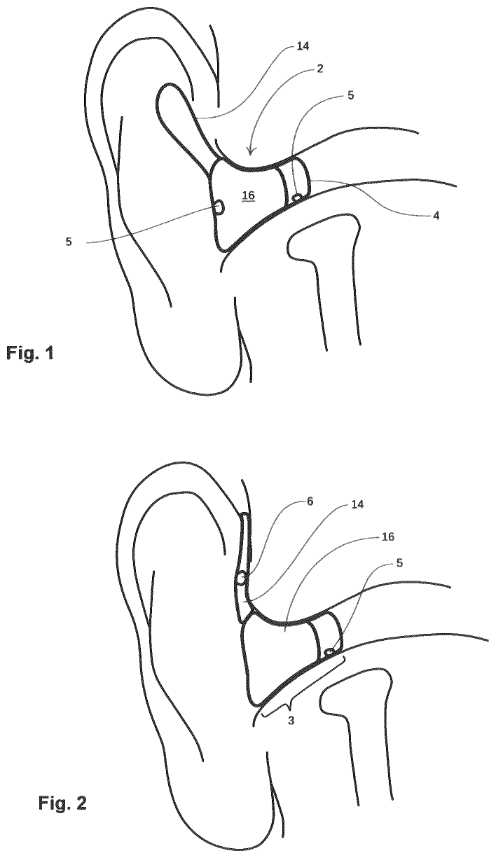

FIG. 1 shows a schematic view of an embodiment of the heart rate sensor 1 for determining a heart rate. The heart rate sensor 1 comprises an in-ear device 2 having an in-ear portion 3 with a distal portion 4 and a proximal portion 16. The distal portion 4 is configured to contact an ear canal of the user when the in-ear device 2 is inserted into the ear canal. The proximal portion 16 is configured to extend towards an exit of the ear canal when the in-ear device 2 is inserted into the ear canal. The proximal portion 16 may seal the ear-canal and/or the proximal portion 16 may provide support for the in-ear device 2 such that the distal portion remains in contact with the ear canal when the in-ear device 2 is placed into the ear canal.

The in-ear device 2 of the heart rate sensor 1 may be configured to be provided in a left ear canal, a right ear canal, or both a left and a right ear canal. The heart rate sensor 1 may further comprise two in-ear devices 2, one for being provided in a left ear canal and one for being provided in a right ear canal.

The distal portion 4 is made from a flexible material such that the distal portion 4 moves, e.g. changes shape, in response to a deformation or vibration of the ear canal when the in-ear device 2 is placed in the ear canal. For example, the flexible material may be shape conforming material such that the shape of the distal portion 4 conforms to the shape of the ear canal as the ear canal moves in response to pulse waves in arteries near the ear canal.

The in-ear device 2 further comprises a magnet 5 and a magnetic sensor 6 configured for measuring a change in magnetic flux density caused by movement of the magnet 5 relative to the magnetic sensor 6. Either the magnet 5 or the magnetic sensor 6 is provided in the distal portion 4 of the in-ear device 2 such that a movement of the distal portion 4 causes a change in the magnetic flux density measured by the magnetic sensor 6. For example, the other one of the magnet 5 and the magnetic sensor 6 may be provided in the proximal portion 16 of the in-ear portion 3 such that there is relative movement between the magnet 5 and magnetic sensor 6 when a deformation or vibration of the ear canal occurs. The other one of the a magnet 5 and magnetic sensor 6 may also be provided in a different portion of the in-ear device 2, e.g. different from the distal portion 4, such as in a support portion 14 of the in-ear device 2 designed for supporting the in-ear device 2 in the ear canal. The magnetic sensor 6 may be a hall effect sensor.

The heart rate sensor 1 further comprises a processor 7 connected to the magnetic sensor 6 for receiving a signal therefrom representing the measured change in magnetic flux density. The processor 7 may be provided in the in-ear device 2. The processor 7 may also be provided in a separate device, such as a smartphone, smartwatch, or a dedicated device. The processor 7 is configured to:
determine a movement of the magnet 5 relative to the magnetic sensor 6 based on the measured change in magnetic flux density;
determine a heart rate of the user, e.g. by first determining a heart beat waveform of the user, based on the determined movement of the magnet 5 relative to the magnetic sensor 6; and
output a signal representing the determined heart rate of the user.

The output signal may be an electronic signal that may be stored in a memory device such that the heart rate of the user may be stored. The output signal may further be a visual signal displayed on a display or an audio signal emitted by an audio device. The output signal may represent the determined heart rate in beats per minute or the output signal may represent the heart rate in any other suitable way.

In embodiments, the processor 7 may be configured to determine the heart rate of the user based on a computer model, which may be based on an empirical model or a simulation model that relates the measured change in magnetic flux to the heart rate, e.g. by first determining a heart beat waveform of the user. In embodiments, the processor 7 is configured to determine the heart rate or the heart beat waveform of the user based on the determined movement of the magnet 5 relative to the magnetic sensor 6 by using an empirical model or a regression model modeling the relation between the heart rate and the determined movement.

In embodiments, the processor 7 may be configured to determine the heart beat waveform from the change in magnetic flux density to determine additional features of the heart rate of the user. These additional features may comprise determining of an anacrotic phase of the heart rate which is related to a rising edge of the heart beat waveform, or a determining of a catacrotic phase which is related to a falling edge of the heart beat waveform. These phases may be linked to the systole phase and diastole phase of the heart beat cycle. In further embodiments, the processor 7 may be configured to determine the dicrotic notch, which is a dip after the systolic peak and before the diastolic peak, that represents the moment that the aortic valve of the heart closes.

FIG. 2 shows a schematic view of another embodiment of a heart rate sensor 1 wherein the magnet 5 is provided in the distal portion 4 and wherein the magnetic sensor 6 is provided in the support portion 14 of the in-ear device 2.

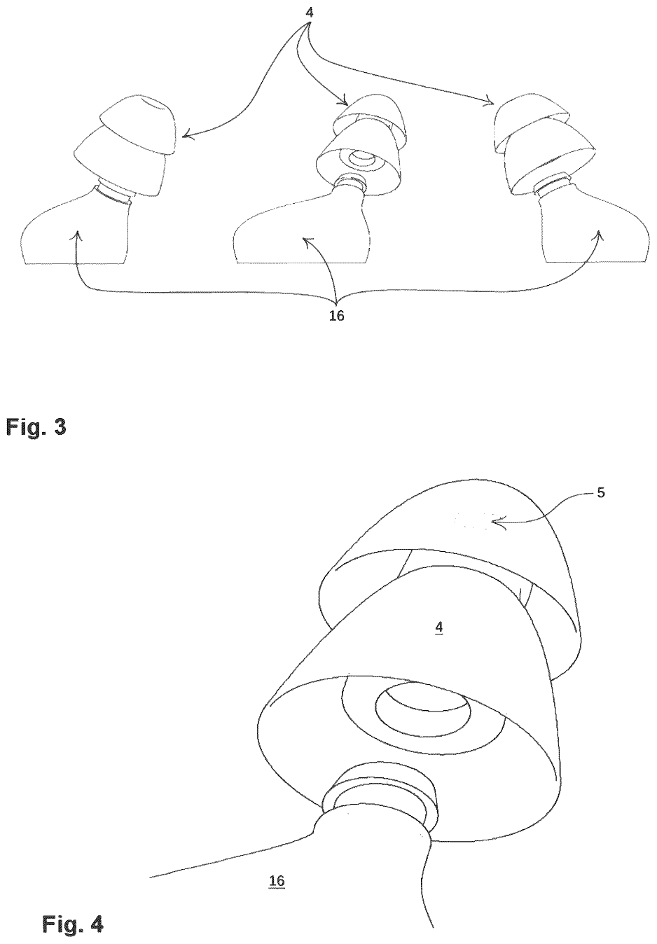

FIG. 3 shows an embodiment of the in-ear portion 3 from various sides, wherein the in-ear portion comprises the distal portion 4 and the proximal portion 16. As before, the magnet 5 or the magnetic sensor 6 is provided in the first distal portion 4 which is made from a flexible material and the other one of the magnet 5 and the magnetic sensor 6 is placed in the proximal portion 16. The shape of the in-ear portion may depend on whether or not the in-ear portion 3 is designed to be placed in a left ear canal or a right ear canal.

In embodiments, the processor 7 is configured to determine the heart rate or the heart beat waveform of the user based on the determined movement of the magnet 5 relative to the magnetic sensor 6 by obtaining a waveform signal from the determined movement of the magnet 5 relative to the magnetic sensor 6 and comparing the waveform signal to a cardiac cycle.

In further embodiments, the processor 7 is further configured to:
determine a movement of the magnet 5 relative to the magnetic sensor 6 based on the measured change in magnetic flux density in an x-direction, y-direction, and/or z-direction of the ear canal;
determine the waveform signal in the x-direction, y-direction, and/or z-direction;
determine a cardinal direction of the waveform signal based on the determined waveform signal in the x-direction, y-direction, and/or z-direction; and
compare the waveform signal in the cardinal direction to the cardiac cycle.

Generally, the relative movement of the magnet 5 with respect to the magnetic sensor 6 may be a three-dimensional movement requiring the device 1 to determine the waveform signal in more than 1 direction to allow for better determination of the heart rate given anatomical and usage variations.

In embodiments, the processor 7 is determined to determine the cardinal direction of the waveform signal by determining a direction of the waveform signal where an amplitude of the waveform signal is above a threshold value. The in-ear device 2 may be placed in the ear-canal by the user in slightly different ways each time, causing a dominant movement of the magnet 5 relative to the magnetic sensor 6 to be different each time. As a result, it may be needed for the processor to determine a cardinal direction in x, y, z space along which the relative movement of the magnet 5 is sufficient, i.e. above the threshold value, to allow for better determining of the heart rate. Similarly, the anatomy of different users may be different requiring the processor 7 to determine the cardinal direction for each user. In practice this may mean that for different users, or different uses, of the device the cardinal direction may be along an x-direction of the ear canal a first time and along a y-direction a second time.

FIG. 4 shows a close-up of the distal portion 4 of the in-ear device 2. As can be seen in the figure, the distal portion 4 may be removable from the rest of the in-ear portion 3, e.g. from the proximal portion 16. For example, this allows the distal portion 4, which is in most contact with the ear canal, to be more easily cleaned or replaced. The magnet 5 or magnetic sensor 6 is provided in the first distal portion 4. There may also be a break between the distal portion 4 and the rest of the in-ear device 2 that allows for better relative movement of the distal portion 4 in relation to the rest of the in-ear device 2. For example, the in embodiments in-ear device 2 comprises a middle portion provided between the distal portion 3 and a proximal portion 16, wherein the other one of the magnet 5 or the magnetic sensor 6 is provided in the proximal portion 16, and wherein the middle portion is configured to allow independent movement of the distal portion 3 relative to the proximal portion 16.

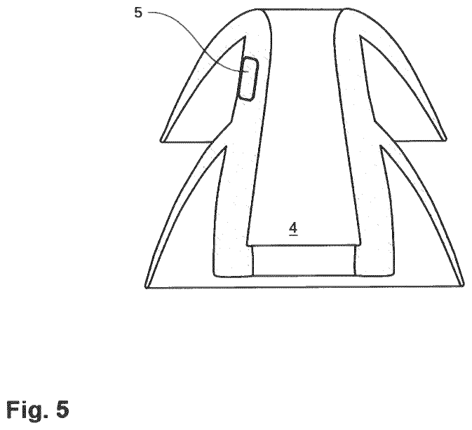

As shown in FIG. 5, which shows a cross-section of a distal portion 4, the distal portion 4 comprises a magnet 5. The magnet 5 may be embodied as a strip running over a portion of the inner surface of the distal portion 4, which allows the magnetic sensor 6 to measure relative movement of the magnet 5 due to changes in the shape of the ear canal. The shape and size of the magnet 5 and the measurement capabilities of the magnetic sensor 6 may allow for more precise measurements, e.g. allow for measurements in multiple dimensions.

In embodiments, processor 7 is configured to obtain the waveform signal by filtering the waveform signal from the determined movement of the magnet 5 relative to the magnetic sensor 6. For example, the sensor device 1 further comprises a movement sensor for measuring motion artifacts caused by movement of the in-ear device 2, wherein the movement sensor is operatively connected to the processor 7 for providing a signal to the processor 7 representative of the measured motion artifacts, wherein the processor 7 is configured to:
remove the motion artifacts from the waveform signal.

In embodiments, the processor 7 is further configured to determine one or more of an inter-beat interval, a heart rate variability, a derivative of the heart rate variability, and spectral signals of the heart rate variability based on the determined movement of the magnet 5 relative to the magnetic sensor 6, and to output a signal representing the determined one or more of the inter-beat interval, the heart rate variability, the derivative of the heart rate variability, and spectral signals of the heart rate variability.

In embodiments, the flexible material of the distal portion 3 may be a suitable, e.g. biocompatible, material that is configured to support the magnet 5 or the magnetic sensor 6 in the ear canal and to conform the shape thereof to the shape of the ear canal.

In embodiments, the in-ear device 2 further comprises a support portion 14 which, when the in-ear device is placed in the ear canal, extends out of the ear canal to support the in-ear device on the ear.

1. A heart rate sensor for determining a heart rate, comprising:
an in-ear device comprising:
an in-ear portion configured to contact an ear canal of an user wherein the in-ear portion comprises a distal portion made from a flexible material such that the distal portion moves, e.g. changes shape, in response to a deformation or vibration of the ear canal when the in-ear portion is placed in the ear canal;
a magnet;
a magnetic sensor for measuring a change in magnetic flux density caused by movement of the magnet relative to the magnetic sensor; and
a processor connected to the magnetic sensor for receiving a signal representing the measured change in magnetic flux density;
wherein either the magnet or the magnetic sensor is provided in the distal portion such that a movement of the distal portion resulting from a movement or vibration of the ear canal causes a change in the magnetic flux density measured by the magnetic sensor,
wherein the processor is configured to:
determine a movement of the magnet relative to the magnetic sensor based on the measured change in magnetic flux density;
determine a heart rate of the user, e.g. by first determining a heart beat waveform of the user, based on the determined movement of the magnet relative to the magnetic sensor; and
output a signal representing the determined heart rate of the user.

2. The heart rate sensor according to claim 1, wherein the processor is configured to determine the heart rate or the heart beat waveform of the user based on the determined movement of the magnet relative to the magnetic sensor by obtaining a waveform signal from the determined movement of the magnet relative to the magnetic sensor and comparing the waveform signal to a cardiac cycle.

3. The heart rate sensor according to claim 2, wherein the processor is further configured to:
determine a movement of the magnet relative to the magnetic sensor based on the measured change in magnetic flux density in an x-direction, y-direction, and/or z-direction of the ear canal;
determine the waveform signal in the x-direction, y-direction, and/or z-direction;
determine a cardinal direction of the waveform signal based on the determined waveform signal in the x-direction, y-direction, and/or z-direction; and
compare the waveform signal in the cardinal direction to the cardiac cycle.

4. The heart rate sensor according to claim 3, wherein the processor is determined to determine the cardinal direction of the waveform signal by determining a direction of the waveform signal where an amplitude of the waveform signal is above a threshold value.

5. The heart rate sensor according to claim 1, wherein the processor is configured to determine the heart rate or the heart beat waveform of the user based on the determined movement of the magnet relative to the magnetic sensor by using an empirical model or a regression model modeling the relation between the heart rate and the determined movement.

6. The heart rate sensor according to claim 2, wherein the processor is configured to obtain the waveform signal by filtering the waveform signal from the determined movement.

7. The heart rate sensor according to claim 6, wherein the sensor further comprises a movement sensor for measuring motion artifacts caused by movement of the in-ear device, wherein the movement sensor is operatively connected to the processor for providing a signal to the processor representative of the measured motion artifacts, wherein the processor is configured to:
remove the motion artifacts from the waveform signal.

8. The heart rate sensor according to claim 1, wherein the in-ear device comprises a middle portion provided between the distal portion and a proximal portion, wherein the other one of the magnet or the magnetic sensor is provided in the proximal portion, and wherein the middle portion is configured to allow independent movement of the distal portion relative to the proximal portion.

9. The heart rate sensor according to claim 1, wherein the processor is further configured to determine one or more of an inter-beat interval, a heart rate variability, a derivative of the heart rate variability, and spectral signals of the heart rate variability based on the determined movement of the magnet relative to the magnetic sensor, and to output a signal representing the determined one or more of the inter-beat interval, the heart rate variability, the derivative of the heart rate variability, and spectral signals of the heart rate variability.

10. The heart rate sensor according to claim 1, wherein the magnetic sensor is a hall effect sensor.

11. The heart rate sensor according to claim 1, wherein the flexible material is a suitable biocompatible material that is configured to support the magnet or the magnetic sensor in the ear canal and to conform the shape thereof to the shape of the ear canal.

12. The heart rate sensor according to claim 1, wherein the in-ear device further comprises a support portion which, when the in-ear device is placed in the ear canal, extends out of the ear canal to support the in-ear device on the ear.

13. A method for determining a heart rate wherein use is made of the heart rate sensor according to claim 1.

14. The method according to claim 13, wherein the method comprises:
determining a movement of the magnet relative to the magnetic sensor based on the measured change in magnetic flux density;
determining a heart rate of the user, e.g. by first determining a heart beat waveform of the user, based on the determined movement of the magnet relative to the magnetic sensor; and
outputting a signal representing the determined heart rate of the user.

15. The method according to claim 14, wherein the method further comprises:
determining the heart rate of the user based on the determined movement of the magnet relative to the magnetic sensor by obtaining a waveform signal from the determined movement of the magnet relative to the magnetic sensor; and
comparing the waveform signal to a cardiac cycle.

16. The method according to claim 15, wherein the method further comprises:
determining a movement of the magnet relative to the magnetic sensor based on the measured change in magnetic flux density in an x-direction, y-direction, and/or z-direction of the ear canal;
determining the waveform signal in the x-direction, y-direction, and/or z-direction;
determining a cardinal direction of the waveform signal based on the determined waveform signal in the x-direction, y-direction, and/or z-direction; and
comparing the waveform signal in the cardinal direction to the cardiac cycle.

17. The method according to claim 16, wherein the method further comprises determining the cardinal direction of the waveform signal by determining a direction of the waveform signal where an amplitude of the waveform signal is above a threshold value.
